(19) United States
(12) Patent Application Publication
(10) Pub. No.: US 2024/0189624 A1
(43) Pub. Date: Jun. 13, 2024

(54) DOSE COVERAGE MARGIN METRIC FOR EVALUATING RADIOTHERAPY TREATMENT PLANS

(71) Applicant: RefleXion Medical, Inc., Hayward, CA (US)

(72) Inventors: Peter Demetri OLCOTT, Los Gatos, CA (US); Angela Jane DA SILVA, Danville, CA (US); Shiyu XU, San Ramon, CA (US); Lingxiong SHAO, Saratoga, CA (US)

(21) Appl. No.: 18/424,533

(22) Filed: Jan. 26, 2024

Related U.S. Application Data
(63) Continuation of application No. PCT/US2022/037293, filed on Jul. 15, 2022.
(60) Provisional application No. 63/227,784, filed on Jul. 30, 2021.

(51) Int. Cl. A61N 5/10; G16H 20/40

(52) U.S. Cl. CPC A61N 5/1075; A61N 5/1039; A61N 5/1071; G16H 20/40; A61N 2005/1041; A61N 2005/1076; A61N 2005/1095

(57) ABSTRACT

Disclosed herein are methods and dose metrics for evaluating the performance and/or quality of a radiotherapy treatment plan and/or radiotherapy system. Dose metrics may be used in a quality assurance (QA) procedure to evaluate (e.g., test, confirm, validate, etc.) the quality of a treatment plan and/or radiotherapy system before radiation is delivered to a patient. One example of a dose metric is a coverage margin metric. The coverage margin metric may provide a way to quantify how tumor motion affects dose coverage and facilitate the determination of whether the tumor contour defined in the treatment plan has a margin that is sufficient to compensate for tumor motion and/or positional changes during radiation therapy.

### CROSS-REFERENCE TO RELATED APPLICATION

This application is a continuation of International Patent Application No. PCT/US2022/037293, filed Jul. 15, 2022, which claims priority to U.S. Provisional Patent Application No. 63/227,784 filed Jul. 30, 2021, the disclosure of which is hereby incorporated by reference in their entirety.

### STATEMENT REGARDING FEDERALLY SPONSORED RESEARCH OR DEVELOPMENT

This invention was made in part during work supported by grant number 5R44CA228753-03 from the National Cancer Institute. The government may have certain rights in the invention.

### BACKGROUND

Radiation therapy aims to deliver a prescribed dose of radiation to a tumor while limiting radiation to surrounding healthy tissue. A radiotherapy treatment plan defines a radiation fluence map that attains clinical goal(s) while limiting irradiation of non-target and/or radiation-sensitive structures. A radiotherapy treatment plan is generated based on planning images (e.g., CT, MR, PET images) acquired before treatment delivery. These planning images provide information on the size, shape, and location of the tumor and radiation-sensitive structures (also referred to as organs at risk or OARs) and are used to define the outlines of the tumor and the OARs (referred to as contours). A clinician can assign dose requirements for each contoured structure and the radiotherapy treatment planning system generates a fluence map that delivers radiation that best meets those dose requirements. The planned radiation fluence map is then translated into a set of radiotherapy system machine instructions so that the calculated fluence map can be delivered to the patient.

However, because there are some position and location uncertainties, the radiation dose delivered to a target region may not match the radiation dose intended by the treatment plan. For example, there may be some position errors due to the imprecision of patient positioning, where the patient's treatment position is not exactly aligned with the patient's position when the planning images were acquired. In addition, the target region may move during the treatment session (e.g., intrafraction motion), and/or may have changed its position between planning and treatment. To account for these position and motion uncertainties and/or errors, the tumor and/or OAR contours may be expanded. For example, tumor contours may be expanded to help ensure that the tumor is irradiated with the prescribed dose regardless of its position and/or motion. The extent of this additional margin added during treatment planning can be subjective, and in cases where the position/motion of a tumor changes between planning and treatment, the added margin may be insufficient to ensure that the prescribed dose is delivered to the tumor. Accordingly, it is desirable to evaluate a treatment plan and delivery system to confirm the dose coverage to a tumor prior to a treatment session, especially when position and motion uncertainties may be significant factors.

### SUMMARY

Disclosed herein are methods and dose metrics for evaluating the performance and/or quality of a radiotherapy treatment plan and/or a radiotherapy system. In one variation, dose metrics for evaluating the quality of a treatment plan and radiotherapy system include one or more of a dose coverage metric and/or a coverage margin metric.

One variation of a method for evaluating a radiotherapy treatment plan for irradiating a target region may comprise acquiring radiotherapy system measurements while operating a radiotherapy system according to a radiotherapy treatment plan, generating a test margin using the acquired radiotherapy system measurements, calculating a coverage margin metric value by calculating a difference between the test margin and a plan margin surrounding a target region, and generating a notification that indicates whether the coverage margin metric value is within an acceptable range. The test margin may be an iso-dose boundary. The plan margin may be an iso-dose boundary that surrounds the target region. A dose level of the iso-dose boundary may be from about 95% to about 105% of a prescribed dose. The test margin may be a boundary that defines a three-dimensional volume. The plan margin may be a boundary that encloses a motion envelope of the target region. The target region may have a margin that encloses a motion envelope of a tumor. In some variations, operating the radiotherapy system may comprise emitting radiation to a phantom. Alternatively, or additionally, operating the radiotherapy system may comprise emitting radiation to a radiation fluence measurement device. The radiation fluence measurement device may comprise one or more an ion chamber, diode array, and/or film. The fluence measurement device may be movable by a motion apparatus according to a motion waveform when radiation is emitted according to the radiotherapy treatment plan. The method may further comprise selecting the motion waveform from a library of motion waveforms. The library of motion waveforms may comprise one or more of a respiratory motion waveform, a peristaltic motion waveform, a breath hold waveform, lateral shift waveform, and any combination thereof.

In some variations, determining the difference between the test margin and a plan margin may comprise calculating distance differences between points along the test margin and the plan margin, and selecting a largest distance difference as the coverage margin metric value. The acceptable range may have an upper threshold value, and generating a notification may comprise determining whether the coverage margin metric value is less than or equal to the threshold value. The upper threshold value may be determined at least in part by a distance between the plan margin and the target region. Optionally, some methods may comprise generating a second notification indicating whether to modify the radiotherapy treatment plan. Modifying the radiotherapy treatment plan may comprise modifying a margin around the target region. In some variations, the radiotherapy system may comprise a therapeutic radiation source, a beam-shaping assembly located in a beam path of the therapeutic radiation source, an MV detector located opposite the therapeutic radiation source, and a motion system configured to move one or more of the therapeutic radiation source, the beam-shaping assembly and the MV detector, and operating the radiotherapy system may comprise emitting radiation (from the therapeutic radiation source), and the acquired radiotherapy system measurements may comprise data from the therapeutic radiation source, the beam-shaping assembly, the MV detector, and/or the motion system. Alternatively, or additionally, operating the radiotherapy system may comprise operating the beam-shaping assembly and the motion system without activating the therapeutic radiation source.

Another variation of a method for evaluating a radiotherapy treatment plan may comprise generating a test margin based on a simulation of radiation emitted according to a treatment plan, calculating a coverage margin metric value by determining a difference between the test margin and a plan margin, and generating a notification that indicates whether the coverage margin metric value is within an acceptable range. The test margin may be a boundary that defines a three-dimensional volume. The plan margin may be an iso-dose boundary that surrounds the target region. In some variations, the plan margin may be a boundary that encloses a motion envelope of the target region. Alternatively, or additionally, the target region may have a margin that encloses a motion envelope of a tumor. A simulation of emitted radiation may comprise calculating a delivery radiation fluence based on simulation models of a radiation beam, a target region, and radiotherapy system components, and generating the test margin may comprise generating a dose distribution based on the calculated delivery radiation fluence and an anatomical image, and generating an iso-dose boundary on the dose distribution. Alternatively, or additionally, a simulation of emitted radiation may comprise calculating a delivery radiation fluence based on imaging data of a target region acquired after generating the treatment plan, and generating the test margin may comprise generating a dose distribution based on the calculated delivery radiation fluence and an anatomical image, and generating an iso-dose boundary on the dose distribution. The imaging data may be generated from a shift of the anatomical image. A dose level of the iso-dose boundary may be from about 95% to about 105% of a prescribed dose.

In some variations, determining the difference between the test margin and a plan margin may comprise calculating distance differences between points along the test margin and the plan margin, and selecting a largest distance difference as the coverage margin metric value. The acceptable range may have an upper threshold value and generating a notification may comprise determining whether the coverage margin metric value is less than or equal to the threshold value. The upper threshold value may be determined at least in part by a distance between the plan margin and the target region. Some variations may further comprise generating a second notification indicating whether to modify the treatment plan. Modifying the treatment plan may comprise modifying a margin around the target region.

Also disclosed herein is a method for determining whether to deliver a radiotherapy treatment plan using newly acquired imaging data. One variation may comprise acquiring imaging data of a patient, generating a dose distribution by calculating a delivery radiation fluence using the acquired imaging data, generating a test margin using the generated dose distribution, calculating a coverage margin metric value by determining a difference between the test margin and a plan margin, and generating a notification that indicates whether the coverage margin metric value is within an acceptable range for delivering radiation according to the radiotherapy treatment plan. The acquired imaging data may comprise one or more PET imaging data, MR imaging data, CT imaging data, and/or X-ray imaging data. Calculating the delivery radiation fluence may comprise convolving a shift-invariant firing filter with the acquired imaging data, where the shift-invariant firing filter is calculated based on a previously-acquired image of the target region. Generating the dose distribution may comprise multiplying a dose calculation matrix with the calculated radiation fluence. The test margin may be a boundary that defines a three-dimensional volume. Generating the test margin may comprise generating an iso-dose boundary on the dose distribution. A dose level of the iso-dose boundary may be from about 95% to about 105% of a prescribed dose. The plan margin may be an iso-dose boundary that surrounds the target region. The plan margin may be a boundary that encloses a motion envelope of the target region. In some variations, the target region may have a boundary that encloses a motion envelope of a tumor.

In some variations, determining the difference between the test margin and the plan margin may comprise calculating distance differences between points along the test margin and the plan margin, and selecting a largest distance difference as the coverage margin metric value. The acceptable range may have an upper threshold value, and generating a notification may comprise determining whether the coverage margin metric value is less than or equal to the threshold value. The upper threshold value may be determined at least in part by a distance between the plan margin and the target region. Some variations may further comprise generating a second notification indicating whether to modify the treatment plan. Modifying the treatment plan may comprise modifying a margin around the target region. In some variations, acquiring imaging data may occur during a previous radiotherapy treatment session, and the generated notification may further comprise a recommendation for updating the treatment plan if the coverage margin metric value is outside of the acceptable range.

One variation of a method for evaluating a radiotherapy treatment plan for irradiating a target region may comprise measuring radiation emitted according to a radiotherapy treatment plan, generating a radiation plot of the measured radiation, generating a test coverage margin mask on the radiation plot by modifying a planned coverage margin mask, calculating a coverage margin metric value by determining a difference between the test coverage margin mask and the planned coverage margin mask, and generating a notification that indicates whether the coverage margin metric value is within an acceptable range. Generating the test coverage margin mask may comprise iteratively modifying the boundaries of the planned coverage margin mask until it encloses voxels of the radiation plot having at least a selected dose level. In some variations, generating the test coverage margin mask may comprise expanding boundaries of the planned coverage margin mask to encompass voxels of the radiation plot having the same dose level. Alternatively, or additionally, generating the test coverage margin mask may comprise contracting boundaries of the planned coverage margin mask to encompass voxels of the radiation plot having the same dose level. The selected dose level may be from about 95% to about 100% of a prescribed dose. The planned coverage margin mask and the test coverage margin mask may be three-dimensional volumes. In some variations, the planned coverage margin mask may be a volume that encloses a motion envelope of a target region.

In some variations, the measured emitted radiation may be emitted to a phantom. Alternatively, or additionally, measured emitted radiation may be emitted to a radiation fluence measurement device. The radiation fluence measurement device may comprise one or more an ion chamber, diode array, and/or film. In some variations, the method may further comprise generating a second notification indicating whether to modify the treatment plan. Modifying the radiotherapy treatment plan may comprise modifying the planned coverage margin mask. One or more of any of the methods described herein may be performed during a QA session where radiation is emitted solely to a phantom and/or radiation fluence measurement device, and is not emitted to a patient.

### BRIEF DESCRIPTION OF THE DRAWINGS

FIG. 1A is a schematic depiction of contours and margins that may be defined during treatment planning.

FIG. 1B is a schematic depiction of a target region, added margins and contours for a target region.

FIG. 1C is a schematic depiction of a target region and a test margin.

FIG. 1D is a schematic depiction of a target region and a test margin.

FIG. 1E is a schematic depiction of a dose coverage margin and the coverage margin metric.

FIG. 2A is a flowchart representation of one variation of a method for calculating a cover margin metric.

FIG. 2B is a flowchart representation of one variation of a method for calculating a cover margin metric.

FIG. 2C is a flowchart representation of another variation of a method for calculating a cover margin metric.

FIG. 3A is a flowchart representation of one variation of a method for evaluating the performance and/or quality of a radiotherapy treatment plan.

FIG. 3B is a flowchart representation of one variation of a method for evaluating the performance and/or quality of a radiotherapy treatment plan.

FIG. 4 is a flowchart representation of one variation of a method for evaluating the performance and/or quality of a radiotherapy treatment plan.

FIG. 5 is a flowchart representation of one variation of a method for evaluating whether a radiotherapy treatment plan is appropriate for delivery on the day of a treatment session.

FIG. 6 is a flowchart representation of one variation of a method where the performance and/or quality of a treatment plan is evaluated at least once before treatment delivery and optionally during treatment delivery.

### DETAILED DESCRIPTION

Disclosed herein are methods and dose metrics for evaluating the performance and/or quality of a radiotherapy treatment plan and radiotherapy system. Dose metrics may be used in a quality assurance (QA) procedure to evaluate (e.g., test, confirm, validate, etc.) the quality of a radiotherapy system before radiation is delivered to a patient. That is, QA procedures may be used to verify whether clinical goals would be met if radiation were to be delivered according to the treatment plan. Existing dose metrics may provide an adequate evaluation of a dose distribution for target regions that are assumed to be static with little or no motion but may not provide a complete assessment of a dose distribution for moving target regions. For example, a gamma index comprising gamma criteria (i.e., a percent dose difference DD and a distance-to-agreement DTA) may be calculated for points in a dose distribution to provide a sense of dose homogeneity and/or how well the delivered dose matches the planned dose. However, the gamma index does not convey how motion and/or positional variabilities affect the quality and/or robustness of a treatment plan to deliver a planned dose. Similarly, dose volume histograms (DVHs) provide a representation of tumor dose coverage, but they do not provide information on how tumor motion and/or positional variabilities affect the dose coverage. That is, gamma index values and/or a DVH for a delivered dose may indicate that a prescribed dose was favorably delivered to a target region, however, these dose metrics may not readily provide an indication as to the extent to which motion and/or position variabilities impacted the dose coverage. For example, gamma index values and/or a DVH on their own do not provide geometric characteristics of the motion impact on dose coverage, e.g., changes in a size of the dose profile or coverage in one dimension (e.g., in mm), two dimensions (e.g., in mm²), and/or in three dimensions (e.g., in mm³). Moreover, these dose metrics do not reveal whether the treatment plan is robust against further motion and/or position variabilities. For example, the treatment plan may have met the dose metric criteria strictly from a dose metric thresholding standpoint, but may have barely “passed”, and further variabilities (especially those introduced by a patient and not precisely mimicked by a QA phantom set up or simulation) may result in an unfavorable delivered dose distribution in the patient.

The coverage margin metric disclosed herein provides a way to quantify how tumor motion affects dose coverage in the reference frame of the tumor and may facilitate the determination of whether the tumor contour defined in the treatment plan has a margin that is sufficient to compensate for tumor motion and/or positional changes during radiation therapy. Moreover, the coverage margin metric may be calculated in the reference frame of the target (as opposed to the radiotherapy system or patient anatomy frame), which facilitates direct visualization of the effect of motion on the target. If the coverage margin metric from a QA session is not within an acceptable range (e.g., if the margin reduction or loss is greater than a threshold value), the margin metric value may help guide the modification (e.g., adjusting, adapting, updating) of the treatment plan by, for example, providing a geometric directive to adjust the margin around the tumor in one dimension (e.g., in mm), two dimensions (e.g., in mm²), and/or in three dimensions (e.g., in mm³) to ensure coverage of the tumor. Adjusting the margin may comprise enlarging or expanding the margin. In some variations, modifying the treatment plan may comprise including an alternative or additional motion management technology. For example, modifying the treatment plan may comprise changing a respiratory-gated stereotactic body radiation therapy (SBRT) plan to a biology-guided radiation therapy (BgRT) plan or a radiation therapy plan that is based on an implanted fiducial. BgRT is a radiation delivery modality that uses PET emission data acquired during a treatment session to deliver radiation that tracks the location of a target region(s). In some variations, dose metrics for evaluating the quality of a radiotherapy treatment plan may include one or more of a dose coverage metric, a coverage margin metric, and a dose volume histogram (DVH) metric. These dose metrics may be used in one or more QA procedures before and/or during a treatment session. For example, a pre-treatment session QA procedure may comprise emitting radiation according to the radiotherapy treatment plan (e.g., to a phantom and/or a fluence measurement device), measuring the emitted radiation (e.g., using a fluence measurement device), calculating a coverage margin metric value (alone or in combination with other dose metrics, such as coverage metric, DVH) based on the radiation measurements, and generating a notification if the coverage margin metric value is not within an acceptable range. Alternatively, or additionally, a pre-treatment session QA procedure may comprise simulating the emission of radiation according to the radiotherapy treatment plan, calculating a coverage margin metric value (alone or in combination with other dose metrics, such as coverage metric, DVH) based on the simulated emitted radiation, and generating a notification if the coverage margin metric value is not within an acceptable range. In some variations, a QA procedure may be performed at the beginning of a treatment session and the dose metrics (e.g., coverage margin metric) from the QA procedure may be used to determine whether to proceed with radiation delivery to the patient. Optionally, QA procedures may be performed during radiation delivery, and dose metrics (e.g., coverage margin metric) may be calculated based on imaging data and/or radiation measurement data acquired during the treatment session.

In some variations, radiotherapy systems may have one or more imaging systems and/or radiation detectors, which may be used for monitoring the performance and/or quality of the radiation delivered to the patient. For example, a radiotherapy system may comprise a therapeutic radiation source, one or more beam-shaping assemblies located in the beam path of the therapeutic radiation source, and an MV detector (e.g., a portal imager comprising an EPID) located opposite the therapeutic radiation source. Some radiotherapy systems may further comprise a CT imaging system, and optionally, a plurality of PET detectors and/or MR sensors. These components may be mounted on one or more gantries or arms whose motion may be controlled by one more motion systems. For example, the CT imaging system and/or PET detectors and/or MR sensors may be located on the same gantry as therapeutic radiation source or on a separate gantry from the therapeutic radiation source. The CT imaging system and/or PET detectors and/or MR sensors may have an imaging field-of-view or imaging plane that is co-planar with the treatment beam plane, or they may have an imaging plane that is not co-planar with the therapeutic radiation beam plane. In one variation, the radiotherapy system may comprise a circular rotatable gantry, a CT imaging system mounted on the gantry, a therapeutic radiation source and MV detector mounted on the gantry, and a plurality of PET detectors mounted on the gantry. The therapeutic radiation source and PET detectors may be co-planar while the imaging plane of the CT imaging system is not co-planar with the treatment beam plane. Additional details and description of radiotherapy systems are provided in U.S. Pat. No. 10,695,586, filed Nov. 15, 2017, which is hereby incorporated by reference in its entirety.

During treatment planning, a clinician may define the outlines or contours of tumor(s) (also referred herein as “target region(s)”) and OARs, as well as additional margins around the contours of the tumor(s) and OAR(s). The additional margins account for position and/or motion variabilities, which may arise from (though not limited to) variabilities in patient positioning, patient motion, radiotherapy system component function (e.g., latencies, resolution limits, noise interference, etc.), any target region tracking and/or motion management mechanisms (e.g., fiducial-based target tracking, PET tracer-based target tracking, breath-hold delivery). One aspect in evaluating the performance and quality of a treatment plan may include determining whether the defined contours and margins sufficiently account for position and/or motion uncertainties, and/or whether a treatment plan radiation fluence follows tumor motion as well as desired to meet clinical goals. Contours and/or margins around tumors and/or OARs may be enlarged beyond the anatomical borders of those structures to help ensure the delivery of prescribed dose despite position and/or motion uncertainties.

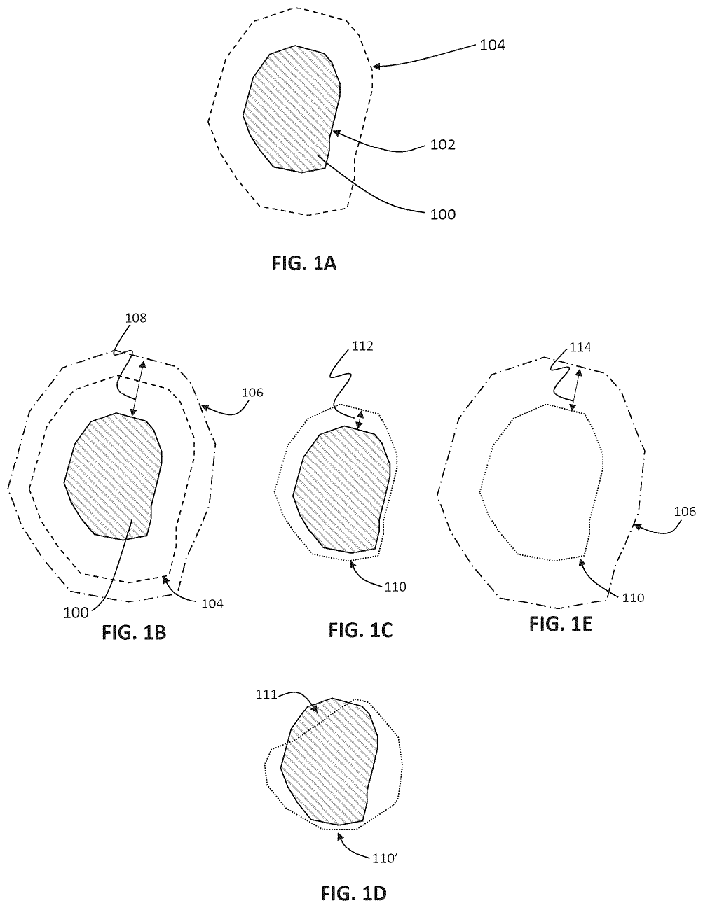

FIG. 1A provides a schematic depiction of contours and margins that may be defined during treatment planning for a target region (with the understanding that similar description applies to OARs). As depicted there, a target region (100) has a contour (102) that may be defined by a clinician. During treatment planning, the clinician may also define an additional margin (104) that surrounds the target region contour (102). The volume enclosed by the added margin (104) is greater than the volume enclosed by the target region contour (102). Although the target region, contours, and margins described and depicted herein are two-dimensional, it should be understood that the target region itself is a three-dimensional volume, and accordingly, its contour and associated margins are three-dimensional boundaries. In some variations, the volume enclosed by a target region contour may enclose one or more of a gross tumor target volume (GTV), clinical target volume (CTV), and/or an internal target volume (ITV). The region enclosed by an added margin contour may additionally enclose one or more of planning target volume (PTV), and/or biology tracking zone (BTZ). One or more of these volumes may include a motion envelope that encompasses the possible locations a target region may occupy during a specified time period (e.g., the possible locations of a tumor during a period of a breathing cycle). For example, the region enclosed by the added margin contour may comprise a PTV that encloses a CTV, where the PTV contour may be an expansion (e.g., about 3 mm to about 10 mm, about 5 mm) of the CTV contour. In some variations, the PTV contour may be the contour of the added margin (104). Alternatively, or additionally, a region enclosed by the added margin contour may comprise a PTV that encloses an ITV that encloses a CTV. The ITV contour may encompass a motion range or envelope of the tumor, while the PTV contour may be an expansion (e.g., about 3 mm to about 10 mm, about 5 mm) of the ITV contour. In some variations, the motion envelope may be accounted for in the definition of the added margin (104), as described above.

FIG. 1B depicts a target region (100), an added margin (104), and a plan margin (106) around the target region (100). The added margin (104) may be geometrically selected to be a boundary that is at a specified distance away from the target region contour or edge. For example, during treatment planning, the added margin (104) may be defined as a boundary that is from about 3 mm to about 10 mm or more, e.g., about 5 mm, away from the target region contour. The plan margin (106) may be an iso-dose boundary at a specified dose level that surrounds the target region contour and the added margin (104). The iso-dose level may be an absolute dose level (e.g., 1.5 Gee, 2 Gy, 5 Gy, etc.), and/or a proportion or percentage of a prescription dose to the target region (e.g., 80% of the dose prescribed to the target region, 90% of the dose prescribed to the target region, 100% of the dose prescribed to the target region, 105% of the prescribed dose to the target region, etc.). For example, the plan margin may be defined as an iso-dose boundary at about 97% of the prescribed dose to the target region. During treatment planning, there may be a margin distance (108) between the target region contour (or edge) and the plan margin (106). The margin distances (108) across the three-dimensional surface of the target region contour may vary (e.g., when the plan margin is an iso-dose boundary, per clinician-specified parameters) or may be constant (e.g., when the plan margin is defined as a boundary having a specified margin distance). A treatment plan fluence map may be generated with the intention or goal of irradiating the entire volume enclosed by the added margin (104) with a prescribed dose of radiation. In this way, even if the target region (100) were to change its position before or during treatment, as long as it remained within the plan margin (106), it would receive an acceptable radiation dose. Alternatively, a treatment plan fluence map may be generated with the intention or goal of irradiating the entire volume enclosed by the target region contour (102) with a prescribed dose of radiation, and may account for motion of the target region within the added margin (104) by tracking the target region location (e.g., using imaging data acquired on the day of treatment, using real-time imaging data acquired during a treatment data, using position sensor data of a fiducial, using breathing motion data as a surrogate for target region motion, etc.).

Treatment Plan QA with Radiation Emission

One or more QA procedures, either in standalone QA sessions or in conjunction with a treatment session, may be performed in order to characterize the delivered dose distribution and evaluate whether radiation delivered according to the treatment plan fluence map would provide the prescribed dose to the target region. In one variation of a QA procedure, the treatment plan fluence map may be delivered to one or more phantom(s) and/or fluence measurement devices using a therapeutic radiation source on a radiotherapy system, and a delivered (or “test”) dose distribution may be generated from radiation emission data (e.g., from a dose chamber associated with a therapeutic radiation source, and/or from the fluence measurement devices). The phantom(s) and/or fluence measurement device(s) may be sized, shaped, and positioned (e.g., moved) to mimic the target region(s) in a patient. For example, the phantom(s) and/or measurement device(s) may be placed on a motion apparatus that simulates respiratory motion. Some variations of QA procedures may comprise selecting a motion waveform from a library of motion waveforms for the phantom(s) and/or fluence measurement device(s). The selected motion waveform may be one that approximates the motion of the target region. For example, the library of motion waveforms may include motion waveforms that approximate motion due to respiration, motion due to peristalsis, as well motion waveforms that are a composite of respiratory and peristaltic motion. Any of the motion waveforms may also simulate one or more breath hold moments interspersed with the respiratory and/or peristaltic motion. Some motion waveforms may also simulate motion as a patient shifts and/or twists on the treatment platform. Data pertaining to selected motion waveform may be transmitted to the motion apparatus for controlling the position of the phantom(s) and/or measurement device(s). The phantom(s) may be anthropomorphic, and the measurement device(s) may be positioned within the phantom at location(s) that correspond with the location of the target region(s) and/or OAR(s). In some variations, the phantom may be filled with a substance (e.g., liquid) that mimics the radiation absorption properties of a patient's body. The fluence measurement devices may comprise one or more of a dosimetry film (e.g., radiochromic EBT-XD film), an ion chamber, dosimeter (e.g., an electrometer), diode arrays, and the like. In some variations, a QA procedure may comprise emitting radiation according to the treatment plan fluence map without a phantom or measurement device in the radiotherapy system, acquiring the radiotherapy system measurements and/or data from one or more of the therapeutic radiation source (e.g., pulse sequence, ion chamber measurements), beam-shaping assembly, the gantry (e.g., gantry position), the motion system, the MV detector, patient platform, and/or any on-board imaging systems (e.g., CT system, PET system) during the radiation emission, and using those measurements and/or data to calculate the emitted radiation dose. Dose metrics, such as the dose coverage metric and/or coverage margin metric, may be calculated based on the delivered or emitted dose distribution (which may be considered the test dose distribution) and used to evaluate whether the treatment plan fluence map is acceptable for patient delivery or whether the treatment plan fluence map needs to be modified. Additional description of treatment plan QA methods that use the dose coverage metric and/or coverage margin metric are provided below.

In some variations, a QA session may comprise characterizing the delivered dose distribution to multiple target regions; that is, evaluating whether radiation delivered according to the treatment plan fluence map would provide the prescribed dose to multiple target regions. The treatment plan fluence map may be delivered to phantoms and/or fluence measurement devices positioned at locations that approximate the locations of the multiple target regions. The phantoms and/or fluence measurement devices may be any of those described herein. A delivered (or “test”) dose distribution may be generated from radiation emission data (e.g., from a dose chamber associated with a therapeutic radiation source, and/or from the fluence measurement devices) for each of the target regions. Optionally, a different motion waveform may be selected for each of the multiple target regions, and these different motion waveforms may be applied to the phantoms and/or fluence measurement devices. A motion apparatus (or a plurality of motion devices) may be used to change the positions of the phantoms and/or fluence measurement devices to mimic the motion of each target region. For example, a first target region may be subject to respiratory motion, a second target region may be subject to peristaltic motion, and a third target region may be subject to respiratory motion and require a breath hold. The corresponding QA procedure may comprise selecting a respiratory motion waveform for a first fluence measurement device, selecting a peristaltic motion waveform from a second fluence measurement device, and selecting a respiratory motion with breath hold motion waveform. The motion waveforms may be selected from a library of waveforms, as described previously, where the library of waveforms may include motion waveforms that are a combination or composite of motions from multiple source (e.g., respiration and peristaltic motion) or motions from individual sources (e.g., respiration only, peristaltic only, etc.). The motion apparatus(es) may be configured to move the three fluence measurement devices in accordance with the three different motion waveforms while radiation is emitted from the therapeutic radiation source. Dose metrics, such as the dose coverage metric and/or coverage margin metric, may be calculated based on the delivered or test dose distribution based on the measurements from the three fluence measurement devices. The dose metrics for each of the three target regions may then be used to evaluate whether the treatment plan fluence map is acceptable for patient delivery or whether the treatment plan fluence map needs to be modified.

In some variations, a QA procedure or session may comprise operating all components of the radiotherapy system in accordance with a treatment plan, but not emitting radiation from the therapeutic radiation source. For example, the linac may not be activated in the QA procedure while the other radiotherapy components are operating as if radiation were being emitted from the linac. A delivered (or “test”) dose distribution may be generated from radiotherapy system measurements, for example, measurements and/or any data from a dose chamber associated with the therapeutic radiation source, one or more beam-shaping assemblies (e.g., jaw position(s), multi-leaf collimator leaf position sensors, collimator aperture sizes and shapes, etc.), a motion system, and/or a patient platform, etc.

Treatment Plan QA with Simulated Radiation Emission

In another variation of a QA procedure, instead of physically delivering radiation using a therapeutic radiation source, the treatment plan fluence map is “delivered” in a simulation environment that may comprise models of the patient, radiation beam, and any physical aspects of the radiotherapy system that impacts the simulated treatment beam. The simulated radiation emission may be combined with anatomical models to generate a delivered (or “test”) dose distribution. In the “virtual” QA procedure with simulated radiation delivery, the patient model (along with models of the target region(s)) may be moved to mimic the motions of the patient and/or the target region(s). Dose metrics, such as the dose coverage metric and/or coverage margin metric, may be calculated based on the delivered or test dose distribution and used to evaluate whether the treatment plan fluence map is acceptable for patient delivery or whether the treatment plan fluence map needs to be modified. The simulated radiation emission may be performed at the time of treatment planning to estimate coverage margin metric that is needed for a given scenario. For example, a tumor may move in a sinusoidal motion with a 10 mm amplitude relative in the superior-inferior direction of the patient. The margin lost from this motion may be calculated by calculating the coverage margin metric from the dose in the target moving reference frame. Additional description of treatment plan QA methods that use the dose coverage metric and/or coverage margin metric are provided below.

### Dose Coverage Metric

In one variation, a QA procedure may comprise calculating a dose coverage metric and/or a coverage margin metric. These dose metrics may provide an indication as to how changes in target region position and/or motion have impacted the delivered dose distribution, and/or information about how well a treatment plan for a particular radiotherapy system delivers radiation dose to a moving target region. The dose coverage metric and the coverage margin metric may be calculated from the dose distribution generated using QA data (e.g., actual delivered radiation and/or simulated radiation delivery). FIG. 1C depicts a schematic representation of the target region (100) and a test margin (110). The contours of the target region (100) may be overlaid with the test dose distribution, and the boundaries of a test margin (110) that corresponds with the plan margin may be defined relative to the target region and/or dose distribution. For example, if a plan margin was selected to be an iso-dose line with a dose level of 97% of the dose prescribed to the target region, the test margin may also be an iso-dose line with a dose level of 97% of the dose delivered to the target region. The dose coverage metric value may be calculated by determining the proportion of the target region that has received a specified proportion of the prescribed dose. For example, the passing criteria for the dose coverage metric may be 100% of the target region receiving at least 97% of the prescribed dose, i.e., the entire target region must receive at 97% or more of the prescribed radiation dose. If the test margin (110) in FIG. 1C is an iso-dose boundary at the dose level of 97% of the prescribed dose, the dose coverage metric value of that QA session may be 100%. FIG. 1D represents another possible scenario, where the test margin (110′) is also a 97% iso-dose boundary, but has a different profile where a portion (111) of the target region is outside of the test margin. In other words, a portion (111) of the target region received less than 97% of the prescribed dose. In the test dose distribution of FIG. 1D, the dose coverage metric value is less than 100%, and therefore, does not meet the passing criteria. Optionally, in variations where the target region (100) comprises a PTV and a CTV within the PTV, additional analyses may be performed to determine whether the dose coverage of the target region (100) includes all of the CTV, even if it excludes portions of the PTV.

### Coverage Margin Metric

The coverage margin metric may facilitate the evaluation of whether a plan margin sufficiently encompasses position and/or motion variabilities. The coverage margin metric represents changes in the margin around the target region; that is, the coverage margin metric represents the difference between a planned dose coverage margin and a test dose coverage margin as calculated based on measurements during a treatment plan QA procedure. The coverage margin metric may indicate whether the dose coverage margin is reduced (i.e., coverage margin loss or reduction) when the planned radiation is delivered to the target region, and may also provide an indication of whether the treatment plan can accommodate further position and/or motion variabilities. FIGS. 1C and 1E depict a schematic representation of a dose coverage margin and the coverage margin metric. As depicted in FIG. 1C, the test margin (110) encloses the target region, which indicates that the target region has been “covered” by the specified dose level. There is a test margin distance (112) between the target region contour (or edge) and the test margin (110). The test margin distance (112) across the three-dimensional surface of the target region contour may vary. Ideally, the test margin distance (112) depicted in FIG. 1C is the same as the plan margin distance (108) depicted in FIG. 1B for each corresponding point along the target region contour, however, the margin distances often differ. In some variations, the test margin distance (112) may be less than the plan margin distance (108), which may be referred to as “margin loss” or “margin reduction”. As depicted in FIG. 1E, the coverage margin metric may be derived from the difference (114) between the plan margin (108) and the test margin (112), as described further below. In some variations, the coverage margin metric may also indicate whether there is any margin gain or expansion. There may be a margin gain or expansion in scenarios where the position and/or motion variability of the target region is less than expected.

A coverage margin metric value that is above a threshold value indicates that the size of the plan margin may not be sufficient for ensuring that a desired dose is delivered to the target region (i.e., there was a relatively large degree of margin loss). For example, a treatment plan that has a coverage margin metric value outside of an acceptable range may not deliver radiation in a way that ensures a desired (e.g., prescribed) dosimetric coverage of a target region. The coverage margin metric may be particularly meaningful in cases where the target region is expected to move substantially before and/or during the treatment session, and may be considered by a clinician as a way to determine whether a particular margin added during treatment planning correctly accounts for target region positional uncertainty and/or motion. For example, if the QA procedure indicates that there was a greater-than-acceptable amount of margin loss or reduction, the clinician may wish to modify the plan by enlarging the added margin (e.g., expanding the PTV) and re-generating the treatment plan fluence map. The dose coverage margin metric may be calculated based on dosimetry measurements of the radiation delivered to a phantom and/or simulated delivery of radiation to a target region. If the value of the coverage margin metric is outside of an acceptable quality and/or performance range (e.g., above a pre-determined threshold value), then a notification may be generated to suggest further analysis and/or treatment plan adjustment so that quality and/or performance criteria are met. In some variations, adjusting the treatment plan may comprise expanding the PTV (i.e., enlarging the added margin around the target region). If the value of the coverage margin metric is within an acceptable range (e.g., below a pre-determined threshold value), then a notification may be generated to indicate that the treatment plan meets the quality and/or performance criteria. In some variations, the coverage margin metric may also indicate whether there is any margin gain or expansion. The margin gain may be considered acceptable up to a threshold quantity, e.g., no more than about 1.5 mm growth, about 2 mm growth, about 3 mm, growth, etc. If the margin gain exceeds the acceptable threshold, a notification may be generated to indicate that treatment plan may need to be adjusted and/or suggest changes to the treatment plan. For example, if the coverage margin gain exceeds an acceptable threshold, the treatment plan may be adjusted by reducing the margin around the target region (e.g., contracting the PTV). Reducing the margin around the target region may help limit the irradiation of surrounding healthy tissue.

In some variations, a planned coverage margin metric value may be the greatest difference in a distance-to-agreement value between the edge of a patient target region (e.g., the contour or boundary of the clinical target volume or CTV) and a plan margin (e.g., iso-dose contour or boundary). The test or delivered coverage margin metric value may be the greatest distance-to-agreement value between the edge of the patient target region and a test or delivered margin (e.g., iso-dose contour or boundary). The coverage margin metric value may be calculated by computing the difference between the planned dose coverage margin and the delivered dose coverage margin. The test coverage margin may be calculated from a radiation sensor (e.g., film, dose chamber, etc.) measurements during a QA procedure where the radiotherapy system delivered radiation according to the treatment plan. Alternatively, or additionally, the test coverage margin may be calculated from a QA simulation of a radiotherapy system that delivered simulated radiation according to the treatment plan. A notification or report may be generated that indicates whether the coverage margin metric is outside an acceptable range. A clinician or technician may use the report to facilitate the evaluation of a treatment plan and generate an updated treatment plan if the previous plan did not meet one or more evaluation criteria. The coverage margin metric value is a scalar (i.e., one-dimensional) value that represents a characteristic of dose delivery to a three-dimensional volume. As such, this dose metric may provide an expeditious way to gauge the effects of motion on dose delivery and guide the modification of a treatment plan.

### Methods for Calculating a Coverage Margin Metric

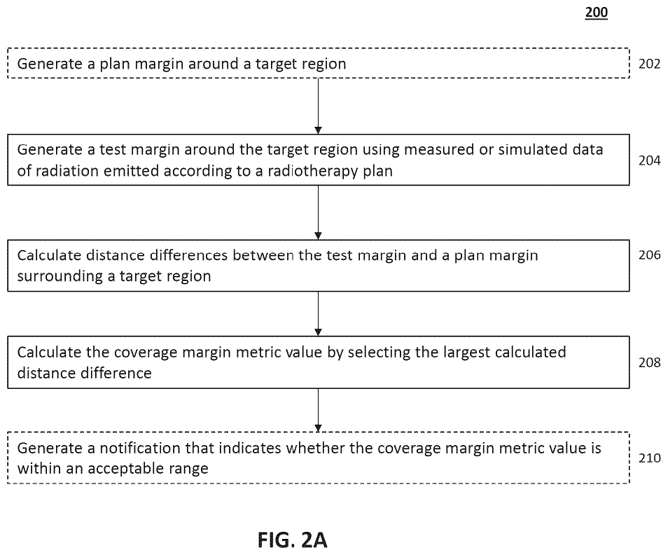

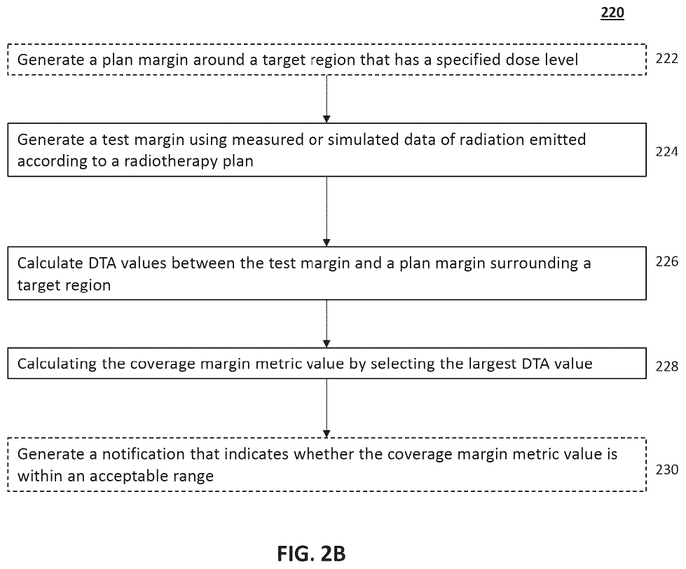

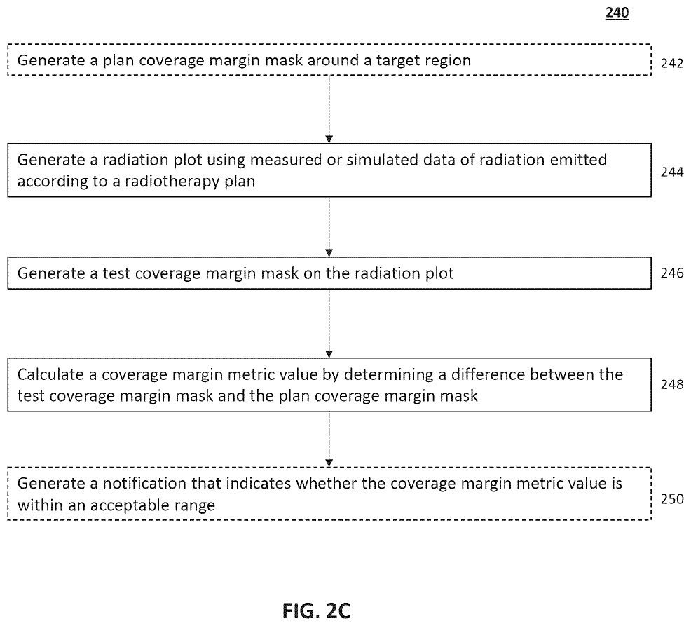

FIG. 2A depicts a flowchart representation of one variation of a method for calculating a cover margin metric during a QA session and/or a treatment session. Method (200) may comprise generating (204) a test margin around a target region using measured or simulated data of radiation emitted according to a radiotherapy plan, calculating (206) distance differences between the test margin and a plan margin, and calculating (208) the coverage margin metric value by selecting the largest calculated distance difference. Optionally, method (200) may comprise generating (202) a plan margin around a target region before generating the test margin. In some variations, the plan margin may be an iso-dose boundary at a specified dose level that surrounds the target region contour. For example, the plan margin may be an iso-dose boundary at a dose level of about 90% or more of the dose prescribed to the target region, e.g., 95% or more of the prescribed dose, 97% or more of the prescribed dose, 99% or more of the prescribed dose, from about 95% to about 105% of the prescribed dose. The plan margin may encompass the PTV contour which is an added margin to the target region contour (e.g., GTV contour, CTV contour, ITV contour). In some variations, the plan margin may user-defined, and the distance between the target region contour and the plan margin may be determined at least in part based on motion data and/or position data. This may help facilitate the definition of a plan margin that encompasses the uncertainties and/or variabilities in target region position and/or motion. In some variations, the PTV contour may encompass a motion envelope of the target region (e.g., motion envelope of a tumor), and the treatment plan may comprise a fluence map that has a plan margin that encompasses the PTV contour.

A QA session may comprise emitting radiation according to the treatment plan, and the radiation delivery may be actual radiation emission or may be simulated (i.e., virtual) radiation emission. In some variations, a QA session may comprise emitting radiation using a therapeutic radiation source of a radiotherapy system to a phantom and/or fluence measurement device, while in other variations, a QA session may comprise simulating the emission of radiation to a virtual model(s) of the target region(s). In either variation, the phantom and/or fluence measurement device and/or virtual model(s) of the target region(s) may emulate the position and/or motion variabilities and/or uncertainties of the actual patient and/or target region(s). Data relating to the actual and/or simulated radiation emission may be used to generate (204) the test margin. In some variations, the test margin may be an iso-dose boundary at a specified dose level, which may be defined as an absolute dose amount (e.g., in Gy) or as a proportion of a dose requirement (e.g., 90% or more of a prescribed dose to a target region). For example, the test margin may be an iso-dose boundary at a dose level of about 90% or more of the dose prescribed to the target region, e.g., 95% or more of the prescribed dose, 97% or more of the prescribed dose, 99% or more of the prescribed dose, from about 95% to about 105% of the prescribed dose. In some variations where the plan margin is an iso-dose boundary at a particular (e.g., selected) dose level, the test margin may also be an iso-dose boundary at the same dose level.

Alternatively, or additionally, in some variations, generating (204) a test margin around a target region may use measured or simulated data from radiotherapy system components. For example, generating (204) a test margin may comprise calculating a dose distribution based on radiotherapy system measurements and/or data from one or more of the therapeutic radiation source (e.g., pulse sequence, ion chamber measurements), beam-shaping assembly, the gantry (e.g., gantry position), the motion system, the MV detector, patient platform, and/or any on-board imaging systems (e.g., CT system, PET system). These measurement data may be used alone or in conjunction with measurements from any fluence measurement devices to generate a “delivered” dose distribution and the test margin. For example, when the QA session does not involve activation of the therapeutic radiation source, a “delivered” dose distribution may calculated using measurements and/or data from the beam-shaping assembly, gantry, motion system, and/or patient platform, but not from the therapeutic radiation source and MV detector. In some variations, radiotherapy system measurements and/or data may be acquired during a QA procedure without any phantoms or fluence measurement devices in the patient area, and the emitted or “delivered” dose distribution may be calculated using the radiotherapy system measurements and/or data.

The test margin and the plan margin each define a three-dimensional volume, and may be registered to each other by aligning the location of the target region in the QA session with the location of the target region in the planning images. The location of the target region during the QA session may be predetermined and/or continuously monitored, and may be aligned with the location of the target region in the planning images. The film holder may have alignment marks that are transferred to the film either using an ink marking or by punching a hole. These alignment marks can be registered in the QA CT image. The planning dose distribution can be interpolated onto the film plane. This defines the static planning dose distribution projected onto the film. The plan margin can be calculated based on the planning dose distribution. The distance differences between the test margin and the plan margin may be calculated for multiple points on the surface of the three-dimensional volumes defined by the planned and test margin boundaries, for example, by finding the shortest distance between a point on the surface of the plan margin boundary and a point on the surface of the test margin boundary. Alternatively, or additionally, the distance differences may be calculated over multiple cross-sections of the three-dimensional volumes (e.g., on a slice-by-slice basis). For example, the volumes defined by the planned and test may be registered, and then sliced into multiple cross-sections, as conceptually depicted in FIG. 1E. For each cross-section, the distance differences may be calculated by finding the shortest distance between a point on the plan margin and a point on the test margin.

Calculating (208) the coverage margin metric value may comprise selecting the largest distance difference out of all of the calculated distance differences across the test margin and the plan margin. The coverage margin metric value may represent the largest degree of change or variance between the tumor position and/or motion at planning and the tumor position and/or motion during the QA procedure, and may provide an indication as to whether the plan margin adequately accounts for dynamic errors from physiological motion, static errors from patient localization (e.g., alignment using CT imaging and/or PET imaging). In addition, the coverage margin metric value may be a one-dimensional (e.g., scalar) value that provides insight to a three-dimensional dose distribution. For example, the units of the coverage margin metric may be a linear dimension, such as millimeters, centimeters, etc., which may facilitate the determination as to whether target region motion and/or position changes have negatively affected the delivered dose distribution.

Method (200) may comprise determining whether the calculated coverage margin metric value is within an acceptable range. If the plan margin sufficiently accounts for the position and motion variabilities of the target region and/or radiotherapy component performance, the calculated coverage margin metric value may be small or close to zero, and the treatment plan may be considered appropriate for patient delivery. However, if the plan margin does not adequately account for the position and motion variabilities of the target region and/or radiotherapy system component performance, the calculated coverage margin metric value may be relatively larger, i.e., larger than an acceptable threshold. In some cases, this may indicate that the test margin has decreased in comparison to the plan margin (i.e., margin loss or reduction) while in other cases, this may indicate that the test margin has increased in comparison to the plan margin (i.e., margin expansion). In either case, the treatment plan may be modified to adjust the margin so that it has better concordance with the likely position and/or motion and/or radiotherapy system variabilities. For example, if the test margin has increased beyond an acceptable threshold, the treatment plan may be modified to reduce the margin around the target region. Method (200) may optionally comprise generating (210) a notification that indicates whether the coverage margin metric value is within an acceptable range. The notification may comprise one or more of an audible, visible, and/or tactile alerts to the user so that they may take appropriate and/or desired actions to adjust the treatment plan. Audible alerts may be output to one or more speakers and visible alerts may be output to one or more display devices (e.g., monitors or screens) of the treatment planning system.

FIG. 2B depicts a flowchart representation of one variation of a method for calculating a cover margin metric during a QA session and/or treatment session. Method (220) may comprise generating (224) a test margin around a target region using measured or simulated data of radiation emitted according to a radiotherapy plan, calculating (226) distance-to-agreement (DTA) values between the test margin and a plan margin, and calculating (228) the coverage margin metric value by selecting the largest DTA value. Optionally, method (220) may comprise generating (222) a plan margin around a target region before generating the test margin. The plan margin and the test margin may be generated as described above with regard to FIG. 2A. Calculating (226) distance-to-agreement (DTA) values between the plan margin and the test margin may comprise finding the distance between a point on plan margin and the nearest point on the test margin with the same dose level. Since the test margin and the plan margin each define a three-dimensional volume, method (220) may comprise calculating DTA values for multiple points along the surfaces defined by the plan margin. Alternatively, or additionally, DTA values may be calculated over multiple cross-sections of the three-dimensional volumes defined by the plan margin and the test margin as described above (e.g., on a slice-by-slice basis). Calculating (228) the coverage margin metric value may comprise selecting the largest DTA value out of all of the calculated DTA values across the test margin and the plan margin, as described above.

Alternatively, or additionally, in some variations, generating (224) a test margin around a target region may use measured or simulated data from radiotherapy system components. For example, generating (204) a test margin may comprise calculating a dose distribution based on radiotherapy system measurements and/or data from one or more of the therapeutic radiation source (e.g., pulse sequence, ion chamber measurements), beam-shaping assembly, the gantry (e.g., gantry position), the motion system, the MV detector, patient platform, and/or any on-board imaging systems (e.g., CT system, PET system). These measurement data may be used alone or in conjunction with measurements from any fluence measurement devices to generate a “delivered” dose distribution and the test margin. For example, when the QA session does not involve activation of the therapeutic radiation source, a “delivered” dose distribution may calculated using measurements and/or data from the beam-shaping assembly, gantry, motion system, and/or patient platform, but not from the therapeutic radiation source and MV detector. In some variations, radiotherapy system measurements and/or data may be acquired during a QA procedure without any phantoms or fluence measurement devices in the patient area, and the emitted or “delivered” dose distribution may be calculated using the radiotherapy system measurements and/or data.

Optionally, the dose metrics described herein may be considered in combination with other dose metrics to evaluate the performance of a treatment plan and/or facilitate the determination of whether to update the treatment plan. In one variation, to evaluate dose accuracy when the target shifts in location during delivery, a method may comprise calculating a dose volume histogram (DVH) of the delivered dose at the completion of the QA procedure, which may include actual radiation emission or simulated radiation emission according to the treatment plan. Calculating the DVH may use the beamlet sequence generated during radiation emission (actual or simulated) to calculate the DVH of the delivered dose. A beamlet sequence may be a dataset that comprises information on patient platform position, gantry position, the multi-leaf collimator (MLC) leaf mask (i.e., leaf configurations) and the radiation pulses fired. The calculated DVH may be compared against a bounded DVH or bDVH calculated during treatment planning. A bDVH may have a lower DVH curve that represents the lowest clinically acceptable dose and an upper DVH curve that represents the highest clinically acceptable dose to a target region. Additional details about the generation of a bDVH may be found in U.S. patent application Ser. No. 16/016,272 filed Jun. 22, 2018, which is hereby incorporated by reference in its entirety. During a QA procedure, the calculated DVH may be evaluated against the bDVH to determine whether it meets specified test criteria. For example, one test criterion may require that 95% of points in the calculated DVHs of all static structures (e.g., the BTZ, OARs) must are within the bounds of the bDVH. Additionally, or alternatively, dose metrics such as the gamma index may be used with one or more of the dose coverage metric, coverage margin metric, and/or DVH to determine whether to modify (e.g., update or re-plan) the treatment plan.

FIG. 2C depicts a flowchart representation of another variation of a method for calculating a cover margin metric during a QA session and/or a treatment session. Method (240) may comprise optionally generating (242) a planned coverage margin mask around a target region. A coverage margin mask may be a one-dimensional line, two-dimensional image, or a three-dimensional volume that encloses the target region with the additional margin. The coverage margin mask may have a contour that is defined by the plan margin boundary that is generated during treatment planning. In some variations, the planned coverage margin mask may comprise a volume that encloses a motion envelope of the target region, e.g., encloses a PTV. Method (244) may comprise generating (244) a radiation plot using measured or simulated data of radiation emitted according to the radiotherapy plan and generating (246) a test coverage margin mask on the radiation plot. In some variations, measuring radiation may comprise emitting radiation using a radiation source (e.g., the therapeutic radiation source of a radiotherapy system) to a radiation fluence measurement device and/or phantom, and acquiring measurements of the emitted radiation. The fluence measurement device may comprise one or more of an ion chamber, diode array, and/or film, as described herein. The radiation plot may comprise a dose distribution over a region that includes the target region. In one variation, the test coverage margin mask may be generated using the planned coverage margin mask. For example, the test coverage margin mask may be generated by overlaying the planned coverage margin mask over the radiation plot and iteratively growing and/or shrinking regions of the planned coverage margin mask according to the radiation dose levels or values of the voxels within the mask. During each iteration, the radiation dose levels or values of voxels within the test coverage margin mask are evaluated to determine whether any of the voxels have dose values that are below a predefined dose threshold level (e.g., a proportion or percentage of the prescribed dose, an absolute dose value). For example, a dose threshold value may be about 95% or more (e.g., about 96% or more, about 97% or more, about 98% or more, about 99% or more, about 100%) of a prescribed dose to the target region. If the dose values are below the predefined threshold, the contour of the test coverage margin mask is adjusted (e.g., moved by expanding outward or drawing inward) toward voxels having dose values that meet or exceed the dose threshold. In some cases, this may involve shrinking (e.g., eroding) the contour of the mask in some cases and expanding (e.g., dilating) the contour of the mask in other cases. The stopping criterion may be met when the voxels within the test coverage margin mask have the dose values that are at or above the predefined threshold. Alternatively, the stopping criterion may be met when a prespecified proportion (e.g., percentage) of the voxels within the test coverage margin mask have dose values at or above the predefined threshold (e.g., 97% of voxels within the test coverage margin mask have dose values above 97% of the prescribed dose to a target region). When the stopping criterion is met, then the iteration stops and the final test coverage margin mask may be compared to the planned coverage margin mask. Method (248) may comprise calculating (248) a coverage margin metric value by determining a difference between the test coverage margin mask and the planned coverage margin mask. The difference may be a number of voxels, and may represent, for example, the number of voxels that have been lost (i.e., margin loss, an eroded number of voxels) or the number of voxels that have been gained (i.e., margin expansion, a dilated number of voxels) between the planned and test coverage margin masks. In other words, if the test coverage margin mask encloses less voxels than the planned coverage margin mask, there is margin loss or erosion. If the test coverage margin mask encloses more voxels than the planned coverage margin mask, there is margin expansion or dilation. Method (240) may comprise determining how much margin loss or gain is acceptable and may optionally comprise generating (250) a notification that indicates whether the coverage margin metric value is within an acceptable range. The acceptable range for the coverage margin metric value may be defined as a maximum number of voxels that can be lost (e.g., maximum coverage margin loss) and the notification may comprise a graphical representation of the number of voxels loss and the acceptable maximum number of voxels that can be lost. Alternatively, or additionally, the acceptable range for the coverage margin metric value may be defined as a maximum proportion (e.g., percentage) of voxel loss, and the notification may optionally comprise a graphical representation of the percentage of voxels lost and the acceptable maximum percentage of voxels that can be lost. Optionally, an acceptable range may comprise a limit on the number and/or percentage of voxels added (i.e., margin expansion). That is, if the test coverage margin mask has expanded too much (i.e., over an acceptable threshold), the notification may comprise a graphical representation of the percentage of voxels gain and the acceptable maximum percentage of voxels that can be gained. In some variations, a second notification (that may appear concurrently with or after a first notification of the coverage margin metric) may comprise a recommendation for modifying the treatment plan.

Alternatively, or additionally, in some variations, generating (244) a test margin around a target region may use measured or simulated data from radiotherapy system components. For example, generating (204) a test margin may comprise calculating a dose distribution based on radiotherapy system measurements and/or data from one or more of the therapeutic radiation source (e.g., pulse sequence, ion chamber measurements), beam-shaping assembly, the gantry (e.g., gantry position), the motion system, the MV detector, patient platform, and/or any on-board imaging systems (e.g., CT system, PET system). These measurement data may be used alone or in conjunction with measurements from any fluence measurement devices to generate a “delivered” dose distribution and the test margin. For example, when the QA session does not involve activation of the therapeutic radiation source, a “delivered” dose distribution may calculated using measurements and/or data from the beam-shaping assembly, gantry, motion system, and/or patient platform, but not from the therapeutic radiation source and MV detector. In some variations, radiotherapy system measurements and/or data may be acquired during a QA procedure without any phantoms or fluence measurement devices in the patient area, and the emitted or “delivered” dose distribution may be calculated using the radiotherapy system measurements and/or data.

In some variations, the dose metric values calculated from data acquired (e.g., measured and/or simulated) during a QA procedure may be used to modify the radiotherapy treatment plan. For example, if the coverage margin metric indicates that there was margin loss or reduction, the radiotherapy treatment plan may be modified to increase the size of the added margin to the target region. In some variations, adjusting the treatment plan may comprise expanding the boundaries/contours of the PTV. The amount of the expansion may be calculated based on the margin loss value; in some variations, the amount of expansion may approximate the margin loss and/or may be greater than the margin loss. For example, if the margin loss or reduction is 2 mm, the treatment plan may be modified so that the PTV boundary is expanded by at least an additional 2 mm. Alternatively, if the coverage margin metric indicates that there was margin expansion, the radiotherapy treatment plan may be modified to reduce the size of the added margin to the target region. Also, if the coverage margin metric fails, it may be an indication that the current motion management strategy may be needed to be changed ensure safe delivery of the plan. For example, a plan that uses gating to manage motion may need to be changed to real-time motion tracking for example. In another example in real-time stereotactic X-ray tracking, if the coverage margin is a function of the latency of the algorithm, the frame rate of the X-ray projection images may need to be increased to lower the coverage margin.

### Quality Assurance Methods for Patient Radiotherapy Treatment Plans

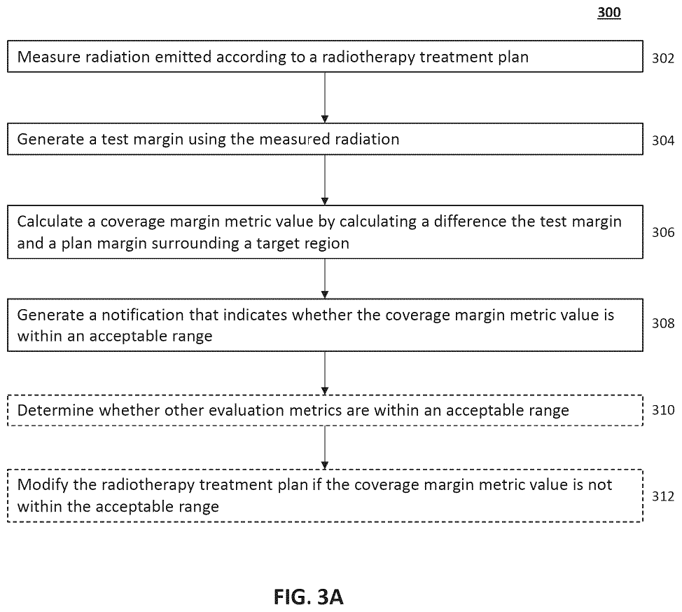

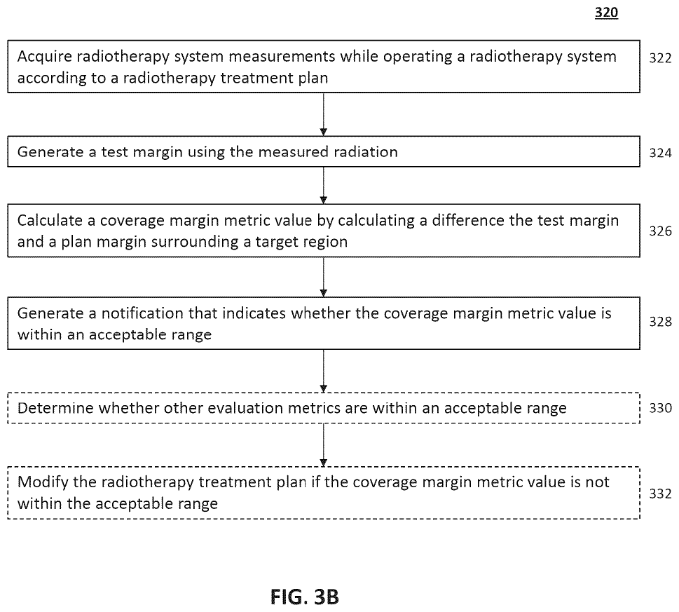

FIG. 3A depicts one variation of a method (300) that may be used to evaluate the performance and/or quality of a radiotherapy treatment plan before radiation delivery. Method (300) may be performed during a QA procedure in a stand-alone QA session (i.e., in the absence of a patient), and/or may be performed at the beginning of a treatment session before therapeutic radiation is emitted to the patient. Method (300) may comprise measuring (302) radiation emitted according to a radiotherapy treatment plan, generating (304) a test margin using the measured radiation, calculating (306) a coverage margin metric value by calculating a difference the test margin and a plan margin, and generating (308) a notification that indicates whether the coverage margin metric value is within an acceptable range. The radiation may be emitted to a phantom and/or fluence measurement devices which may measure (i.e., acquire data on) the emitted radiation. The phantom(s) and/or measurement devices may be any of the phantoms and/or measurement devices described above, including, for example, anthropomorphic phantoms that have various portions with different densities and/or measurement devices positioned at locations that correspond to target region and/or OAR locations. Optionally, the phantom(s) and/or measurement devices may be moving in accordance with selected motion waveforms, as described above. In some variations, radiotherapy system measurements and/or data may be acquired during a QA procedure without a patient or fluence measurement devices in the patient area, and the emitted or “delivered” dose distribution may be calculated using the radiotherapy system measurements and/or data. In some variations, a phantom (such as any described herein) may optionally be present in the patient area. For example, radiotherapy system measurements and/or data may be acquired from one or more of the therapeutic radiation source (e.g., pulse sequence, ion chamber measurements), beam-shaping assembly, the gantry (e.g., gantry position), the motion system, the MV detector, patient platform, and/or any on-board imaging systems (e.g., CT system, PET system). The test margin may be generated (304) and the coverage margin metric value may be calculated (306) from the emitted or “delivered” dose distribution using any of the methods described above in FIGS. 2A-2B. The generated notification may comprise one or more of an audible, visible, and/or tactile alerts to the user so that they may take appropriate and/or desired actions to adjust the treatment plan. Audible alerts may be output to one or more speakers and visible alerts may be output to one or more display devices (e.g., monitors or screens) of the treatment planning system. Optionally, method (300) may comprise determining (310) whether other radiotherapy treatment plan evaluation metrics, e.g., other dose metrics such as dose coverage metric, DVH, and/or gamma index, are within an acceptable range. The result of this determination (310) may be reflected in the generated notification (308). In some variations, method (300) may comprise modifying (312) radiotherapy treatment plan if the coverage margin metric value is not within the acceptable range. The treatment plan may be modified (e.g., updated or re-planned) as described above, and in some variations, the dose metric values calculated during method (300) may be used to guide the adjustments to the treatment plan. Method (300) may be used to calculate dose metric values for multiple target regions.

Alternatively, instead of emitting radiation according to the radiotherapy treatment plan, the radiotherapy system may be operated according to the radiotherapy treatment plan without activating the therapeutic radiation source. This variation is depicted in FIG. 3B. In this variation, generating a test margin may use measured or simulated data from radiotherapy system components. For example, generating a test margin may comprise calculating a dose distribution based on radiotherapy system measurements and/or data from one or more of the beam-shaping assembly, the gantry (e.g., gantry position), the motion system, the MV detector, patient platform, and/or any on-board imaging systems (e.g., CT system, PET system). A “delivered” dose distribution may be calculated using measurements and/or data from the beam-shaping assembly, gantry, motion system, and/or patient platform, but not from the therapeutic radiation source and MV detector (since therapeutic radiation source was not activated). Method (320) may comprise acquiring (322) radiotherapy system measurements while operating a radiotherapy system according to a radiotherapy treatment plan, generating (324) a test margin using the acquired radiotherapy system measurements, calculating (326) a coverage margin metric value by calculating a difference the test margin and a plan margin surrounding a target region, and generating (328) a notification that indicates whether the coverage margin metric value is within an acceptable range. Acquiring (322) radiotherapy system measurements and/or data may comprise acquiring measurements and/or data from one or more of the therapeutic radiation source (e.g., pulse sequence, ion chamber measurements), beam-shaping assembly, the gantry (e.g., gantry position), the motion system, the MV detector, patient platform, and/or any on-board imaging systems (e.g., CT system, PET system). These measurements and/or data may then be used to generate an emitted or “delivered” dose distribution (i.e., the test dose distribution). The test margin may be generated (324) and the coverage margin metric value may be calculated (326) from the emitted or “delivered” dose distribution using any of the methods described above in FIGS. 2A-2B. The generated notification may comprise one or more of an audible, visible, and/or tactile alerts to the user so that they may take appropriate and/or desired actions to adjust the treatment plan. Audible alerts may be output to one or more speakers and visible alerts may be output to one or more display devices (e.g., monitors or screens) of the treatment planning system. Optionally, method (320) may comprise determining (330) whether other radiotherapy treatment plan evaluation metrics, e.g., other dose metrics such as dose coverage metric, DVH, and/or gamma index, are within an acceptable range. The result of this determination (330) may be reflected in the generated notification (328). In some variations, method (320) may comprise modifying (332) radiotherapy treatment plan if the coverage margin metric value is not within the acceptable range. The treatment plan may be modified (e.g., updated or re-planned) as described above, and in some variations, the dose metric values calculated during method (320) may be used to guide the adjustments to the treatment plan. Method (320) may be used to calculate dose metric values for multiple target regions.

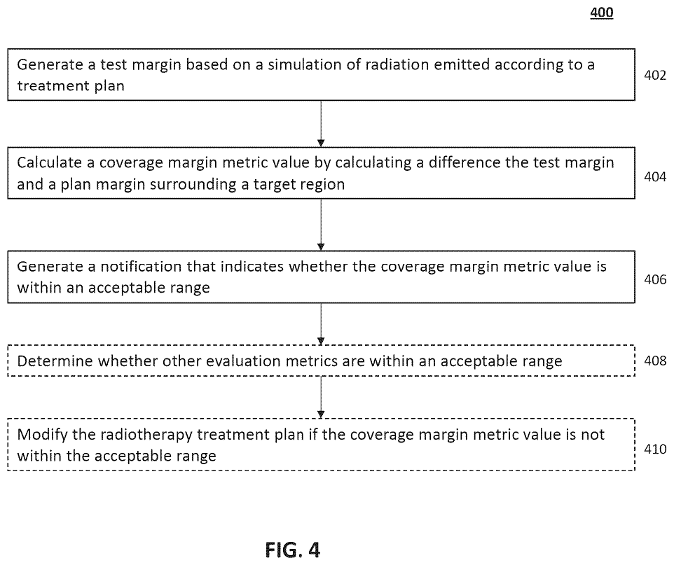

FIG. 4 depicts another variation of a method (400) that may be used to evaluate the performance and/or quality of a radiotherapy treatment plan before radiation delivery, where radiation emission is simulated using various models of the patient, target region(s), OAR(s), beam, radiotherapy system components, etc. Method (400) may be performed during a QA procedure in a stand-alone QA session (i.e., in the absence of a patient), and/or may be performed at the beginning of a treatment session before therapeutic radiation is emitted to the patient. Method (400) may comprise generating (402) a test margin based on a simulation of radiation emitted according to a treatment plan, calculating (404) a coverage margin metric value by calculating a difference the test margin and a plan margin, and generating (406) a notification that indicates whether the coverage margin metric value is within an acceptable range. In some variations, the simulation of emitted radiation may comprise calculating a delivery radiation fluence based on imaging data of a target region acquired after generating the treatment plan, and where generating the test margin comprises generating a dose distribution based on the calculated delivery radiation fluence and an anatomical image, and generating an iso-dose boundary on the dose distribution. The imaging data may be imaging data that is acquired after the treatment planning image (i.e., more recently-acquired imaging data) and/or may be imaging data that is generated by shifting an existing image (e.g., shifting PET imaging data). For example, the imaging data may be generated by shifting an anatomical image. Optionally, the QA procedure may comprise modeling the motion(s) of target region(s) in accordance with selected motion waveforms, as described above. The test margin may be generated (402) using this simulated radiation emission and the coverage margin metric value may be calculated (404) using any of the methods described above in FIGS. 2A-2B. The generated notification may comprise one or more of an audible, visible, and/or tactile alerts to the user so that they may take appropriate and/or desired actions to adjust the treatment plan. Audible alerts may be output to one or more speakers and visible alerts may be output to one or more display devices (e.g., monitors or screens) of the treatment planning system. Optionally, method (400) may comprise determining (408) whether other radiotherapy treatment plan evaluation metrics, e.g., other dose metrics such as dose coverage metric, DVH, and/or gamma index, are within an acceptable range. The result of this determination (408) may be reflected in the generated notification (406). In some variations, method (400) may comprise modifying (410) radiotherapy treatment plan if the coverage margin metric value is not within the acceptable range. The treatment plan may be modified (e.g., updated or re-planned) as described above, and in some variations, the dose metric values calculated during method (400) may be used to guide the adjustments to the treatment plan. Method (400) may be used to calculate dose metric values for multiple target regions.

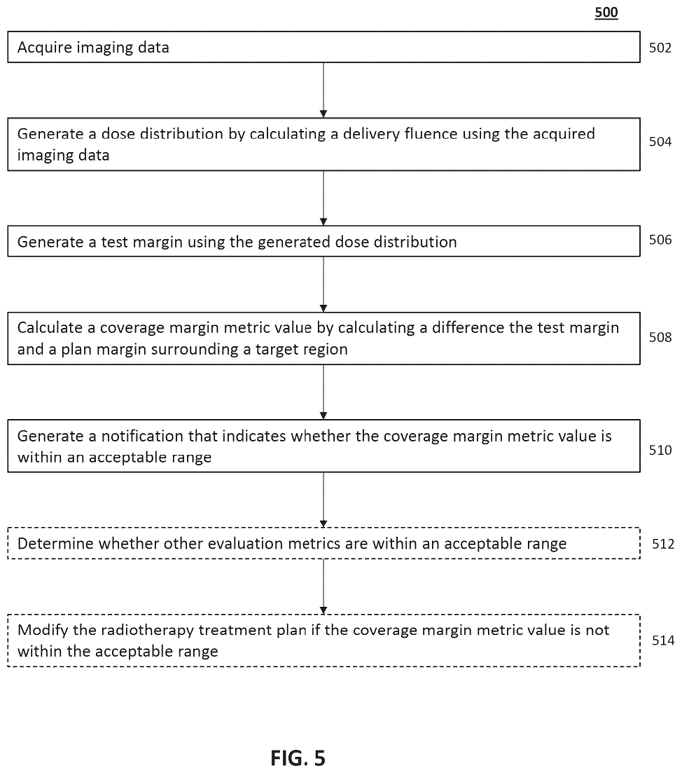

FIG. 5 depicts another variation of a method (500) that may be used to evaluate whether a radiotherapy treatment plan is appropriate for delivery on the day of the treatment session. In some variations, this method may be used in biology-guided radiation therapy (BgRT), which uses PET emission data acquired during a treatment session to deliver radiation that tracks the location of a target region(s). A radiotherapy system configured to delivery BgRT may comprise a therapeutic radiation source (e.g., a linear accelerator or linac) and an imaging system (e.g., PET imaging system, CT imaging system, MR imaging system, X-ray imaging system) that is configured to acquire imaging data during radiation delivery. One variation of a radiotherapy system configured for BgRT may comprise a rotatable gantry, a linac mounted on the rotatable gantry, and a plurality of PET detectors also mounted on the rotatable gantry such that their imaging plane is co-planar with the irradiation plane of the linac. For example, a BgRT system may have two PET detector arcs arranged on the gantry such that they are not in the beam path of the linac. Method (500) may be performed during a QA procedure in a stand-alone QA session (i.e., in the absence of a patient), and/or may be performed at the beginning of a treatment session before therapeutic radiation is emitted to the patient. For example, method (500) may be performed at the beginning of a treatment session using PET and/or CT prescan imaging data. Method (500) may comprise acquiring (502) imaging data, generating (504) a dose distribution by calculating a delivery fluence using the acquired imaging data, generating (506) a test margin using the generated dose distribution, calculating (508) a coverage margin metric value by calculating a difference the test margin and a plan margin, and generating (510) a notification that indicates whether the coverage margin metric value is within an acceptable range. In some examples, imaging data may be positron annihilation emission data acquired using PET detectors that may or may not be in-plane with the therapeutic radiation source. Alternatively, or additionally, imaging data may be MR data acquired using MR sensors and/or CT data acquired using CT detectors that may or may not be in-plane with the therapeutic radiation source.

In BgRT, the radiation fluence for delivery is calculated from the acquired imaging data. The imaging data may be acquired over a short period of time (e.g., less than a couple of seconds, less than about 1 second, about 0.5 sec) and may be referred to as a “partial image”, since it may not contain sufficient imaging data to generate a “full image” that is visually decipherable by a user. For example, the radiation fluence for delivery may be calculated by convolving the acquired imaging data with firing filters that are generated during treatment planning using the planning images. A radiation fluence map may comprise radiation beamlet patterns and/or beamlet intensities (e.g., beamlet weights). A firing filter p can be a vector or a matrix depending on the dimensions of the delivered fluence field. The firing filter facilitates the conversion from image data to a radiation fluence map by convolution. In other words, the firing filter p represents the relationship between a radiation fluence map F and an image X of a target region such that F=p*X, where * denotes a convolution operator. During radiation delivery, partial images may be convolved with the firing filters to produce a fluence map or pattern that is delivered by the linac. Since convolution is a shift invariant operation, if the target region moves during delivery, the fluence map will move correspondingly and facilitate the delivery of radiation dose that effectively “tracks” the target.

Additional details and examples of firing filters (which may be derived from shift invariant radiation firing matrices) and methods of BgRT treatment planning and delivery may be found in U.S. Pat. No. 10,688,320 filed May 30, 2018, which is hereby incorporated by reference in its entirety. The dose distribution may be generated (504) by combining (e.g., multiplying) the calculated radiation fluence with a dose calculation matrix. A dose calculation matrix may represent a dose contribution from a particular beamlet to each of the voxels in a target region, and in some variations, may be generated using anatomical image data (e.g., CT image data, MR image data). In one example, the dose calculation matrix may be a (k×n) matrix where n may be the number of candidate beamlets {bᵢ} and k may be the number of pre-selected voxels for a target region. An i-th column of a dose calculation matrix (which has k elements) may represent a dose contribution from a unity-weighted beamlet bᵢ to each of the k voxels.

The test margin may be generated (506) and the coverage margin metric value may be calculated (508) using any of the methods described above in FIGS. 2A-2B. The generated notification may comprise one or more of an audible, visible, and/or tactile alerts to the user so that they may take appropriate and/or desired actions to adjust the treatment plan. Audible alerts may be output to one or more speakers and visible alerts may be output to one or more display devices (e.g., monitors or screens) of the treatment planning system. Optionally, method (500) may comprise determining (512) whether other radiotherapy treatment plan evaluation metrics, e.g., other dose metrics such as dose coverage metric, DVH, and/or gamma index, are within an acceptable range. The result of this determination (512) may be reflected in the generated notification (510). In some variations, method (500) may comprise stopping (e.g., pausing) radiation delivery and modifying (514) radiotherapy treatment plan if the coverage margin metric value is not within the acceptable range. The treatment plan may be modified (e.g., updated or re-planned) as described above, and in some variations, the dose metric values calculated during method (500) may be used to guide the adjustments to the treatment plan. Method (500) may be used to calculate dose metric values for multiple target regions.

Generating a dose distribution based on imaging data acquired on the day of treatment may provide an updated estimate and/or prediction of the dose that may be delivered in that day's treatment session and facilitate the decision as to whether to proceed with treatment (i.e., whether the delivered dose be safe and/or meet clinical prescriptions). As described above, method (500) may be performed at the beginning of a treatment session, however, in some variations, method (500) may be performed throughout the treatment session, concurrently and/or sequentially with therapeutic radiation delivery. For example, a treatment session may comprise moving the patient platform multiple times through the treatment beam (i.e., in multiple shuttle passes). Imaging data of the target regions may be acquired as the patient is irradiated in a shuttle pass. Method (500) may be performed at the completion of each shuttle pass based on the imaging data acquired during the shuttle pass. The evaluation of the calculated dose distribution using the one or more dose metrics described herein may facilitate the decision whether to continue or stop treatment; that is, whether to proceed with the next shuttle pass or to end the treatment session.

In some variations, the coverage margin metric may be calculated after a radiation delivery session (e.g., after a treatment session) to verify that the radiation delivery met coverage margin metric standards (e.g., within an acceptable range, margin loss did not exceed a predetermined threshold). For example, after a BgRT treatment session, the coverage margin metric (and/or dose coverage metric and/or DVHs) may be calculated to verify that the delivered radiation meets treatment planning standards, i.e., the target regions received the prescribed dose of radiation, and OARs were not over-irradiated. The dose distribution may be generated using the imaging data acquired during the treatment session, and/or may, alternatively or additionally, be generated using delivered beamlet sequence information (e.g., multi-leaf collimator leaf position data, jaw width data, linac pulse sequence, patient platform position, etc.). The dose coverage metric, coverage margin metric, and/or DVHs may be calculated from the post-treatment dose distribution and compared to predetermined acceptable ranges to verify whether the delivered dose distribution met standards. The comparison of the treatment dose metrics with the acceptable ranges and/or standards may facilitate the decision as to whether radiation delivery should be modified for the next treatment session or fraction. For example, after a first treatment session, it may be determined based on the generated dose distribution that the dose coverage metric met the acceptable standard (e.g., the acceptable standard is >97% of target received the prescribed dose and the dose distribution has a dose coverage metric of 99%) but the coverage margin metric did not meet the acceptable standard (e.g., the acceptable standard is less than a 3 mm change in the coverage margin metric, and the dose distribution has a coverage margin metric of −5 mm, i.e., a margin loss or reduction of 5 mm). If coverage margin changes during a treatment session and is no longer within the acceptable range, a notification may be generated that includes a suggestion for modifying the treatment plan.

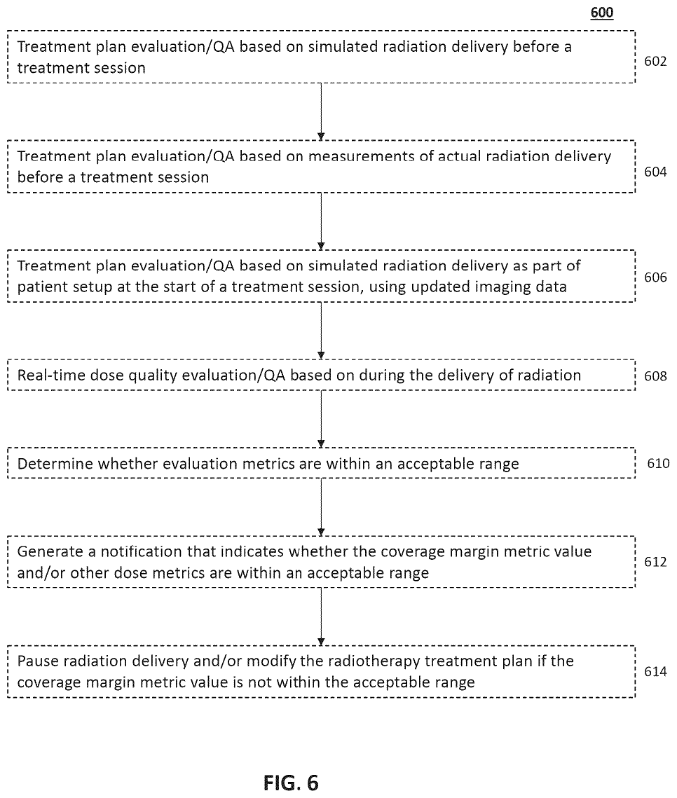

The treatment plan evaluation and/or performance methods described herein may be used in one or more QA procedures before a treatment session and/or during a treatment session. The dose coverage metric and/or coverage margin metric may be used in one or more of the QA procedures. FIG. 6 depicts a flowchart representation of one variation of a method (600) where the performance and/or quality of a treatment plan is evaluated at least once before treatment delivery and optionally during treatment delivery. Method (600) may comprise evaluating (602) the treatment plan based on simulated radiation delivery before a treatment session and/or evaluating (604) treatment plan based on measurements of actual radiation delivery before a treatment session. In some variations, evaluation of a treatment plan based on simulated radiation delivery may be performed as part of the treatment planning workflow and may be performed as described above with regard to FIG. 4. Evaluation of based on measurements of actual radiation delivery may be performed as described above with regard to FIG. 3. Method (600) may optionally further comprise evaluating (606) the treatment plan based on simulated radiation delivery as part of patient setup at the start of a treatment session. This setup evaluation may be performed as described above with regard to FIG. 5 and/or FIG. 4. This evaluation may use updated imaging data that has been acquired at the start of the treatment session, and may be used to decide whether it is safe and/or therapeutic to proceed with treatment. Method (600) may also optionally comprise evaluating (608) the quality of the delivered dose during the delivery of therapeutic radiation delivery; i.e., provide real-time QA of the radiation dose delivery. This real-time QA evaluation may be performed as described above with regard to FIG. 5 and/or FIG. 4.

Optionally, method (600) may comprise determining (610) whether radiotherapy treatment plan evaluation metrics, e.g., dose metrics such as coverage margin metric, dose coverage metric, DVH, and/or gamma index, for any one or more of the evaluations (602-608) are within an acceptable range. The result of this determination (610) may be reflected in a generated notification (612). In some variations, method (600) may comprise stopping (e.g., pausing) radiation delivery and modifying (614) radiotherapy treatment plan if the coverage margin metric value is not within the acceptable range. The treatment plan may be modified (e.g., updated or re-planned) as described above, and in some variations, the dose metric values calculated during method (600) may be used to guide the adjustments to the treatment plan.

While various inventive variations have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments/variations described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive variations described herein. It is, therefore, to be understood that the foregoing variations are presented by way of example only and that, within the scope of the appended claims and equivalents thereto, inventive variations may be practiced otherwise than as specifically described and claimed. Inventive variations of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. A method for evaluating a radiotherapy treatment plan for irradiating a target region, the method comprising:
acquiring radiotherapy system measurements while operating a radiotherapy system according to a radiotherapy treatment plan;
generating a test margin using the acquired radiotherapy system measurements;
calculating a coverage margin metric value by calculating a difference between the test margin and a plan margin surrounding a target region; and
generating a notification that indicates whether the coverage margin metric value is within an acceptable range.

2. The method of claim 1, wherein the test margin is an iso-dose boundary.

3. The method of claim 2, wherein a dose level of the iso-dose boundary is from about 95% to about 105% of a prescribed dose.

4. The method of any one of claims 1-3, wherein the test margin is a boundary that defines a three-dimensional volume.

5. The method of any one of claims 1-3, wherein the plan margin is an iso-dose boundary that surrounds the target region.

6. The method of any one of claims 1-5, wherein the plan margin is a boundary that encloses a motion envelope of the target region.

7. The method of any one of claims 1-6, wherein operating the radiotherapy system comprises emitting radiation to a phantom.

8. The method of any one of claims 1-7, wherein operating the radiotherapy system comprises emitting radiation to a radiation fluence measurement device.

9. The method of claim 8, wherein the radiation fluence measurement device comprises one or more an ion chamber, diode array, and/or film.

10. The method of claim 8, wherein the fluence measurement device is movable by a motion apparatus according to a motion waveform when radiation is emitted according to the radiotherapy treatment plan.

11. The method of claim 10, further comprising selecting the motion waveform from a library of motion waveforms.

12. The method of claim 11, wherein the library of motion waveforms comprises one or more of a respiratory motion waveform, a peristaltic motion waveform, a breath hold waveform, lateral shift waveform, and any combination thereof.

13. The method of any one of claims 1-12, wherein determining the difference between the test margin and a plan margin comprises
calculating distance differences between points along the test margin and the plan margin, and
selecting a largest distance difference as the coverage margin metric value.

14. The method of any one of claims 1-13, wherein the acceptable range has an upper threshold value, and wherein generating a notification comprises determining whether the coverage margin metric value is less than or equal to the threshold value.

15. The method of claim 14, wherein the upper threshold value is determined at least in part by a distance between the plan margin and the target region.

16. The method of any one of claims 1-15, further comprising generating a second notification indicating whether to modify the radiotherapy treatment plan.

17. The method of claim 16, wherein modifying the radiotherapy treatment plan comprises modifying a margin around the target region.

18. The method of any one of claims 1-17, the target region has a margin that encloses a motion envelope of a tumor.

19. The method of any one of claims 1-18, wherein the radiotherapy system comprises a therapeutic radiation source, a beam-shaping assembly located in a beam path of the therapeutic radiation source, an MV detector located opposite the therapeutic radiation source, and a motion system configured to move one or more of the therapeutic radiation source, the beam-shaping assembly and the MV detector, wherein operating the radiotherapy system comprises emitting radiation, and wherein the acquired radiotherapy system measurements comprise data from the therapeutic radiation source, the beam-shaping assembly, the MV detector, and/or the motion system.

20. The method of any one of claims 1-19, wherein the radiotherapy system comprises a therapeutic radiation source, a beam-shaping assembly located in a beam path of the therapeutic radiation source, an MV detector located opposite the therapeutic radiation source, and a motion system configured to move one or more of the therapeutic radiation source, the beam-shaping assembly and the MV detector, and wherein operating the radiotherapy system comprises operating the beam-shaping assembly and the motion system without activating the therapeutic radiation source.

21. A method for evaluating a radiotherapy treatment plan, the method comprising:
generating a test margin based on a simulation of radiation emitted according to a treatment plan;
calculating a coverage margin metric value by determining a difference between the test margin and a plan margin; and
generating a notification that indicates whether the coverage margin metric value is within an acceptable range.

22. The method of claim 21, wherein the simulation of emitted radiation comprises calculating a delivery radiation fluence based on simulation models of a radiation beam, a target region, and radiotherapy system components, and wherein generating the test margin comprises generating a dose distribution based on the calculated delivery radiation fluence and an anatomical image, and generating an iso-dose boundary on the dose distribution.

23. The method of any one of claims 21-22, wherein the simulation of emitted radiation comprises calculating a delivery radiation fluence based on imaging data of a target region acquired after generating the treatment plan, and wherein generating the test margin comprises generating a dose distribution based on the calculated delivery radiation fluence and an anatomical image, and generating an iso-dose boundary on the dose distribution.

24. The method of claim 23, wherein the imaging data is generated from a shift of the anatomical image.

25. The method of any one of claims 22-24, wherein a dose level of the iso-dose boundary is from about 95% to about 105% of a prescribed dose.

26. The method of any one of claims 21-25, wherein the test margin is a boundary that defines a three-dimensional volume.

27. The method of any one of claims 21-26, wherein the plan margin is an iso-dose boundary that surrounds the target region.

28. The method of any one of claims 21-27, wherein the plan margin is a boundary that encloses a motion envelope of the target region.

29. The method of any one of claims 21-28, wherein determining the difference between the test margin and a plan margin comprises
calculating distance differences between points along the test margin and the plan margin, and
selecting a largest distance difference as the coverage margin metric value.

30. The method of any one of claims 21-29, wherein the acceptable range has an upper threshold value, and wherein generating a notification comprises determining whether the coverage margin metric value is less than or equal to the threshold value.

31. The method of claim 30, wherein the upper threshold value is determined at least in part by a distance between the plan margin and the target region.

32. The method of any one of claims 21-31, further comprising generating a second notification indicating whether to modify the treatment plan.

33. The method of claim 32, wherein modifying the treatment plan comprises modifying a margin around the target region.

34. The method of any one of claims 21-33, the target region has a margin that encloses a motion envelope of a tumor.

35. A method for determining whether to deliver a radiotherapy treatment plan, the method comprising:
acquiring imaging data of a patient;
generating a dose distribution by calculating a delivery radiation fluence using the acquired imaging data;
generating a test margin using the generated dose distribution;
calculating a coverage margin metric value by determining a difference between the test margin and a plan margin; and
generating a notification that indicates whether the coverage margin metric value is within an acceptable range for delivering radiation according to the radiotherapy treatment plan.

36. The method of claim 35, wherein the acquired imaging data comprises one or more PET imaging data, MR imaging data, CT imaging data, and/or X-ray imaging data.

37. The method of any one of claims 35-36, wherein calculating the delivery radiation fluence comprises convolving a shift-invariant firing filter with the acquired imaging data, wherein the shift-invariant firing filter is calculated based on a previously-acquired image of the target region.

38. The method of any one of claims 35-37, wherein generating the dose distribution comprises multiplying a dose calculation matrix with the calculated radiation fluence.

39. The method of any one of claims 35-38, wherein generating the test margin comprises generating an iso-dose boundary on the dose distribution.

40. The method of claim 39, wherein a dose level of the iso-dose boundary is from about 95% to about 105% of a prescribed dose.

41. The method of any one of claims 35-40, wherein the test margin is a boundary that defines a three-dimensional volume.

42. The method of any one of claims 35-41, wherein the plan margin is an iso-dose boundary that surrounds the target region.

43. The method of any one of claims 35-42, wherein the plan margin is a boundary that encloses a motion envelope of the target region.

44. The method of any one of claims 35-43, wherein determining the difference between the test margin and the plan margin comprises
calculating distance differences between points along the test margin and the plan margin, and
selecting a largest distance difference as the coverage margin metric value.

45. The method of any one of claims 35-44, wherein the acceptable range has an upper threshold value, and wherein generating a notification comprises determining whether the coverage margin metric value is less than or equal to the threshold value.

46. The method of claim 45, wherein the upper threshold value is determined at least in part by a distance between the plan margin and the target region.

47. The method of any one of claims 35-46, further comprising generating a second notification indicating whether to modify the treatment plan.

48. The method of claim 47, wherein modifying the treatment plan comprises modifying the plan margin.

49. The method of any one of claims 35-48, the target region has a boundary that encloses a motion envelope of a tumor.

50. The method of any one of claims 35-49, wherein acquiring imaging data occurs during a previous radiotherapy treatment session, and the generated notification further comprises a recommendation for updating the treatment plan if the coverage margin metric value is outside of the acceptable range.

51. A method for evaluating a radiotherapy treatment plan for irradiating a target region, the method comprising:
measuring radiation emitted according to a radiotherapy treatment plan;
generating a radiation plot of the measured radiation;
generating a test coverage margin mask on the radiation plot by modifying a planned coverage margin mask;
calculating a coverage margin metric value by determining a difference between the test coverage margin mask and the planned coverage margin mask; and
generating a notification that indicates whether the coverage margin metric value is within an acceptable range.

52. The method of claim 51, wherein generating the test coverage margin mask comprises expanding boundaries of the planned coverage margin mask to encompass voxels of the radiation plot having the same dose level.

53. The method of any one of claims 51-52, wherein generating the test coverage margin mask comprises contracting boundaries of the planned coverage margin mask to encompass voxels of the radiation plot having the same dose level.

54. The method of any one of claims 51-53, wherein generating the test coverage margin mask comprises iteratively modifying the boundaries of the planned coverage margin mask until it encloses voxels of the radiation plot having at least a selected dose level.

55. The method of claim 54, wherein the selected dose level is from about 95% to about 100% of a prescribed dose.

56. The method of any one of claims 51-55, wherein the planned coverage margin mask and the test coverage margin mask are three-dimensional volumes.

57. The method of any one of claims 51-56, wherein the planned coverage margin mask is a volume that encloses a motion envelope of a target region.

58. The method of any one of claims 51-57, wherein the measured emitted radiation is emitted to a phantom.

59. The method of any one of claims 51-58, wherein the measured emitted radiation is emitted to a radiation fluence measurement device.

60. The method of claim 59, wherein the radiation fluence measurement device comprises one or more an ion chamber, diode array, and/or film.

61. The method of any one of claims 51-60, further comprising generating a second notification indicating whether to modify the treatment plan.

62. The method of claim 61, wherein modifying the radiotherapy treatment plan comprises modifying the planned coverage margin mask.
